(19) United States
(12) Patent Application Publication
(10) Pub. No.: US 2014/0288478 A1
(43) Pub. Date: Sep. 25, 2014

(54) BANDAGE PACKAGE AND METHOD FOR USING SAME

(71) Applicant: JR Medical Products, LLC, Dallas, TX (US)

(72) Inventors: Jerry Holubec, Allen, TX (US); Robert McCoskey, Dallas, TX (US)

(21) Appl. No.: 14/298,120

(22) Filed: Jun. 6, 2014

Related U.S. Application Data
(62) Division of application No. 13/793,874, filed on Mar. 11, 2013, now abandoned.

(51) Int. Cl. A61F 15/00

(52) U.S. Cl. CPC A61F 15/002
USPC 602/57

(57) ABSTRACT

A new bandage package having a loosely packaged carrier material with a plurality of bandages attached. The package allows the bandages to be kept sterile when opening the package by allowing the carrier to fall freely to a sterile surface without the user touching the carrier.

### CROSS-REFERENCE TO RELATED APPLICATIONS

This application is a Divisional of U.S. patent application Ser. No. 13/793,874 filed Mar. 11, 2013, which is incorporated herein by reference in its entirety.

### BACKGROUND

1. Field of the Invention

The present invention relates to adhesive bandage packages, and more particularly a packaging system for a plurality of bandages to facilitate removal of the plurality of bandages from the packaging.

2. Description of the Related Art

Adhesive bandages have been used for years to apply to wounds on the human body. They have been used extensively in connection with surgical procedures to cover needle pricks from administering various drugs. In a surgical environment, the medical professionals must take care to keep the environment sterile. One requirement is to wear gloves. When wearing gloves it is difficult to remove traditional bandages from packaging and apply to the human body. To apply the bandage, it is a common occurrence for the medical professional to remove a glove in order to allow the bandage to be more easily applied to the wound. The problem with this is that the person who is applying the bandage is now potentially exposed to blood borne diseases when applying the bandage and it is also easier for the bandage to become contaminated by contact with the finger.

When the glove is removed to open the packaging and apply the bandage, the sterility of the bandage is easily compromised during this process. Moreover, it is often difficult to remove a bandage that has been applied to the human body. In the past, bandages have typically been packaged one at a time for use in an operating room environment rather than in packages containing multiple bandages. This does not present a problem if there is only one puncture to cover, but if there are multiple punctures, it takes significant time to open six or eight bandage packages, for example. Referring now to FIG. 1 and FIG. 2, an example of an adhesive bandage package 100 as it exists currently in the art is illustrated. The bandage package has outer packaging 110, 120 that is peeled apart as shown in FIG. 2 to reveal bandages 130, 140. The bandages 130, 140 are stuck to a backing material and then sandwiched between the outer packaging 110, 120. The user must typically insert a finger nail between the bandages 130, 140 and the backing material to remove the bandages from the backing material. This is difficult to do while wearing gloves.

Adhesive bandage packaging as it currently exists also typically uses excessive packaging material that increases the cost of manufacturing the bandage as well as the corresponding waste. For example, bandages typically have a backing material on the bandage itself and then are wrapped in a wrapper for distribution to the end user.

Thus, there is a need for an improved bandage and packaging system that will allow a medical professional to easily open a bandage package and remove the bandage for application to the patient while wearing gloves. There is a need for bandages that can be more easily removed when they are no longer needed. There is also a need for a packaging design that minimizes waste and is inexpensive to manufacture.

### SUMMARY

Disclosed in this application is a new bandage package having a loosely packaged carrier material with a plurality of bandages attached. The package allows the bandages to be kept sterile when opening the package by allowing the carrier to fall freely to a sterile surface without the user touching the carrier.

### BRIEF DESCRIPTION OF THE DRAWINGS

FIG. 1 is a top view of a prior art adhesive bandage package.

FIG. 2 is a top view of a prior art adhesive bandage packaged in an open configuration.

FIG. 3 is a top view of an opened adhesive bandage package in accordance with an embodiment of the invention.

FIG. 4 is a top view of an opened adhesive bandage package in accordance with an alternate embodiment of the invention.

FIG. 5 is a top view of an opened adhesive bandage package with a separate carrier material in accordance with an alternate embodiment of the invention.

FIG. 6A is a side view showing the joining of an adhesive material and non-adhesive material in accordance with an embodiment of the invention.

FIG. 6B is a top view of a strip showing the joined adhesive material and non-adhesive material in accordance with an embodiment of the invention.

FIG. 7 is a flow diagram showing the application of cushions to the strip in accordance with an embodiment of the invention.

FIG. 8 is a flow diagram showing the application of white paper to the strip in accordance with an embodiment of the invention.

FIG. 9 is a flow diagram showing the cutting of individual bandages from the strip in accordance with an embodiment of the invention.

FIG. 10 is a flow diagram showing the packaging of individual bandages in accordance with an embodiment of the invention.

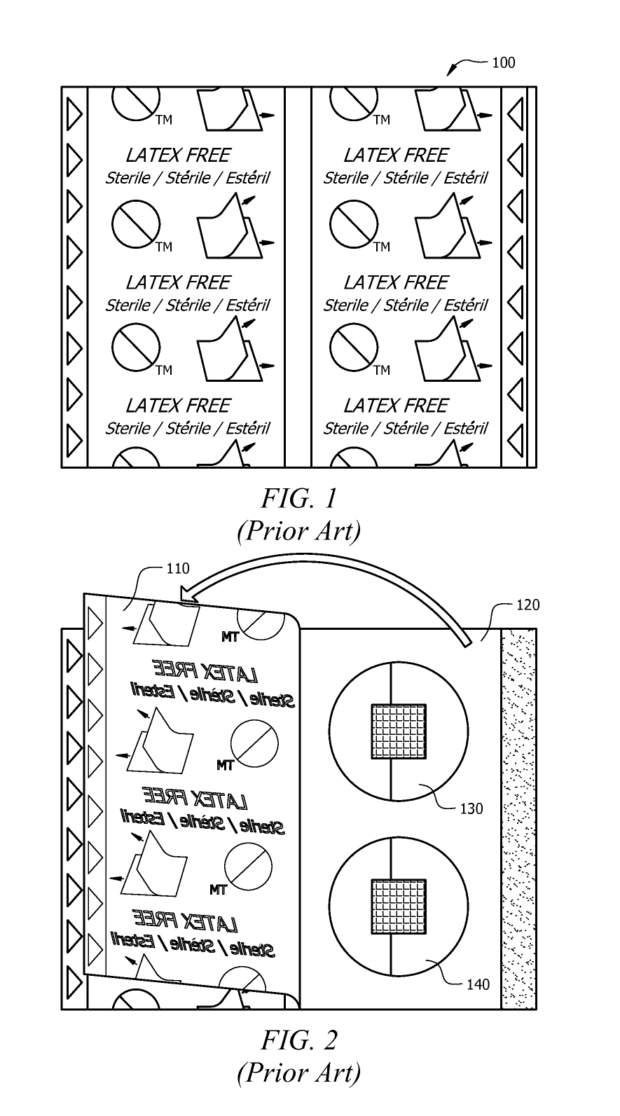

### DETAILED DESCRIPTION

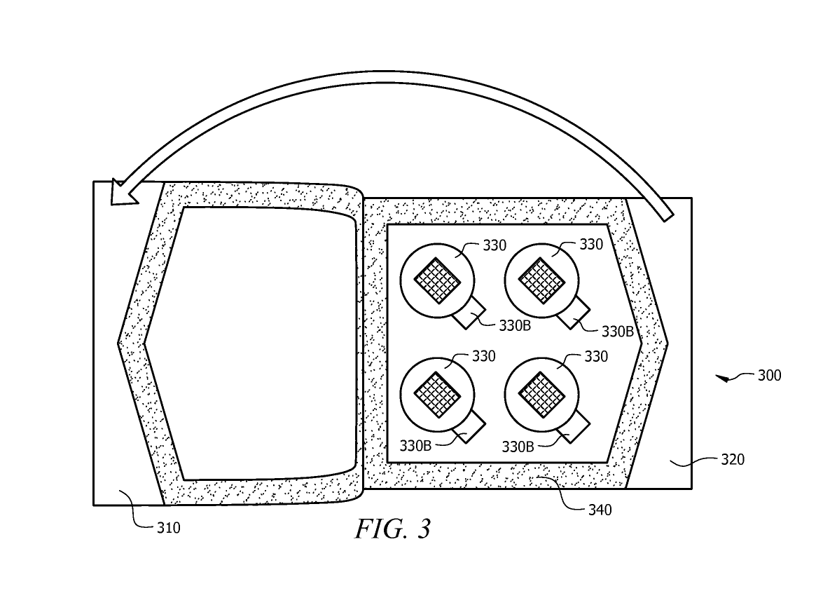

Referring now to FIG. 3, a top diagram of an open bandage package in accordance with an embodiment of the present invention is illustrated. A top outer covering 310 is peeled back from a lower outer covering 320 to expose four bandages 330. Each of the bandages 330 also contains a tab 330B. The tab 330B does not contain any adhesive on the backside of it and thus is not adhered to the lower outer covering 320. However, the adhesive portion of the bandage 330 is adhered to the lower outer covering 320. By using the outer covering 320 of the packaging as a backing material instead of using an additional backing material as is typical with current bandage packages, waste can be avoided and the cost of production decreased.

To remove the adhesive bandages 330, the user simply grabs the tab 330B and pulls the adhesive bandage away from the outer covering 320. Because the tab 330B is not adhered to the lower outer covering 320, the user can easily grab the tab 330B while wearing gloves. The lower outer covering 320 can be secured to the top outer covering 310 with pressure sensitive adhesive in the shaded border area 340 or in any other manner known in the art.

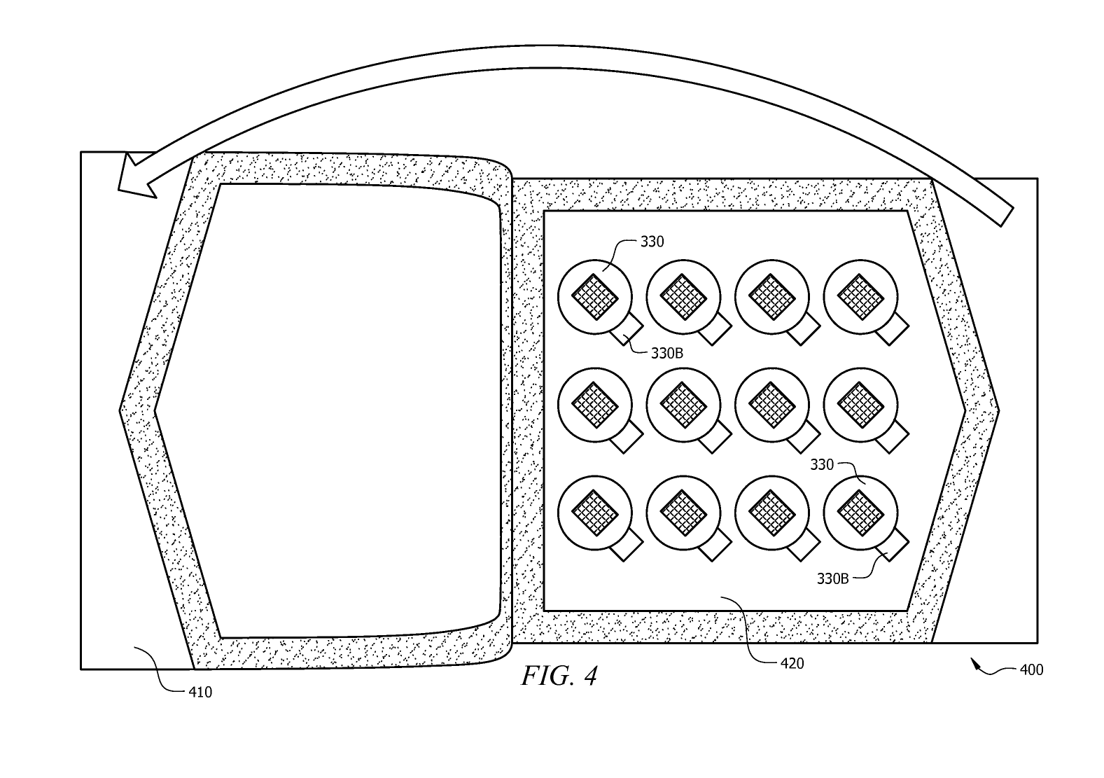

The adhesive bandages 330 with tabs 330B attached can be supplied in multiple configurations. Referring now to FIG. 4, an illustration of second embodiment of an opened adhesive bandage package is illustrated. As shown in FIG. 4, the adhesive bandage package 400 contains twelve adhesive bandages applied to the lower outer covering 420 of the adhesive bandage package 400. The top outer covering 410 is peeled back to expose the twelve adhesive bandages 330. A medical professional can then remove as many bandages as are needed for a particular procedure. In a sterile operating environment when a package such as those identified in FIGS. 3 and 4 are used, the bandage packages cannot be reused in a second procedure if less than all of the bandages are consumed in the first procedure. Otherwise, the sterility of the second operating environment could be compromised. However, the medical professional can choose the size of the bandage package depending on the particular procedure that is being performed and the likely bandages that will be used in that procedure so as to avoid unnecessary waste.

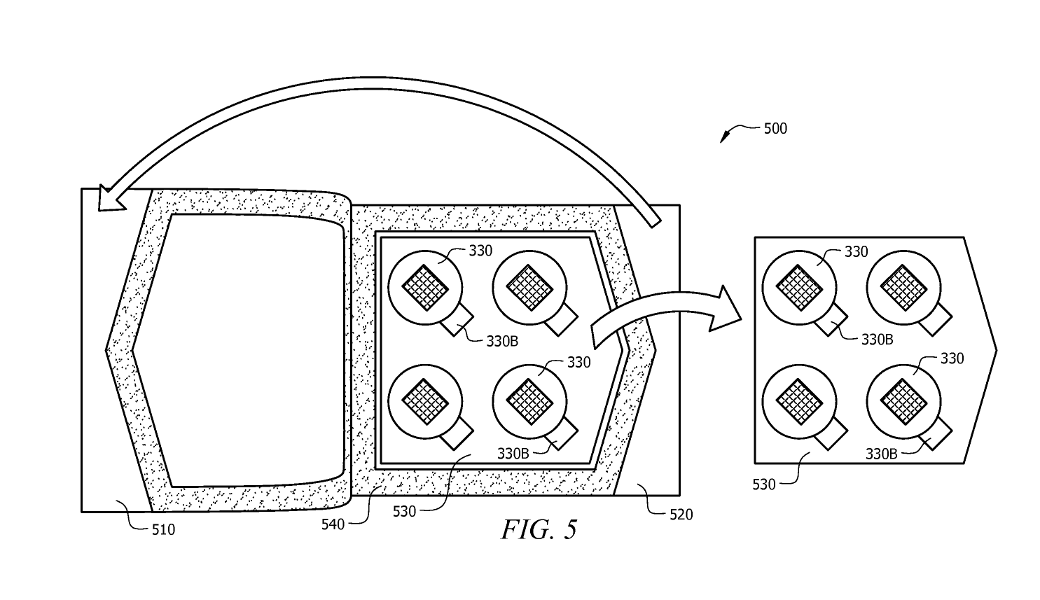

Referring now to FIG. 5, a top view of an open adhesive bandage package in accordance with an alternate embodiment of the invention is illustrated. The adhesive package 500 illustrated in FIG. 5 is constructed in a similar manner to that of the packages shown in FIGS. 3 and 4. However, instead of applying the bandages 330 directly to the lower outer covering 520, the bandages 330 are instead applied to a carrier insert 530. In operation, the user can peel back the top outer covering 510 while holding the bottom outer covering 520 and hold the packaging over a sterile operating tray at the same time. When opened in an inverted position, the sterile carrier insert 530 with the adhesive bandages 330 attached will fall out onto the tray without being touched by the user. It is a common procedure for medical professionals to open packages and drop them on the tray in this manner so as to preserve the sterility of the operating room environment. The user can then remove the adhesive bandages 330 in the same manner as they are removed as discussed with respect to FIGS. 3 and 4 by holding the tab 330B and pulling the bandage from the carrier insert 530.

By using the adhesive bandages described with respect to FIGS. 3, 4, and 5, the medical professional can now easily open the packaging and remove the adhesive bandages and apply to a patient without frustration. This significantly reduces the temptation to remove a glove in order to apply a bandage, thus helping to protect the safety of both the patient and medical professional. Additionally, because the tab is part of the adhesive bandage itself, it can be used to remove the adhesive bandage when the patient is no longer in need of the bandage. This prevents the situation in which a bandage being in place for a significant length of time becomes very difficult to remove from the patient.

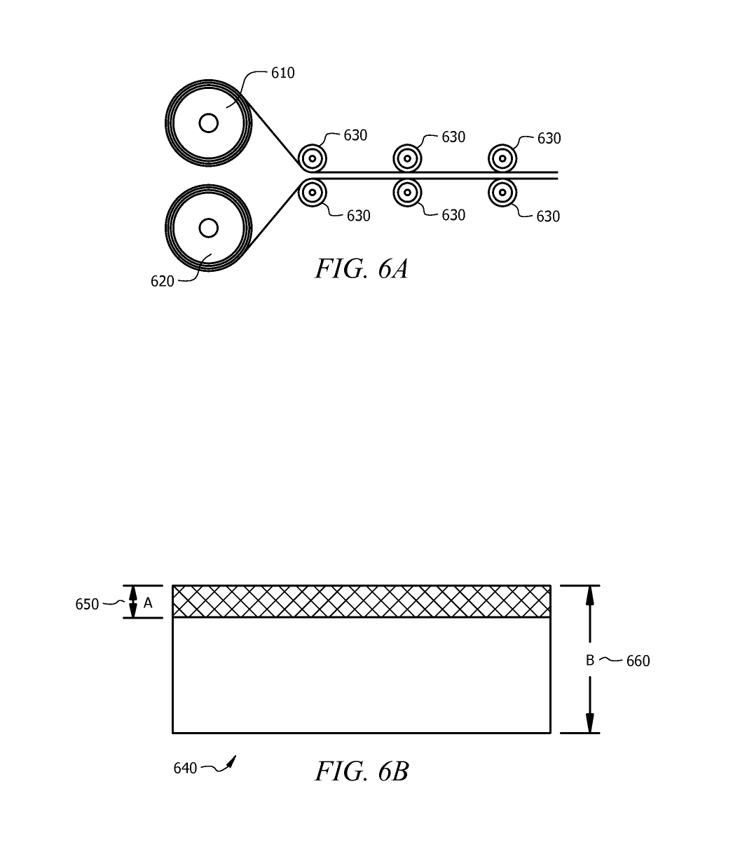

Referring now to FIGS. 6A-10, a method of manufacturing the adhesive bandages in accordance with one embodiment of the invention is illustrated. A roll of non-adhesive fabric 610 is joined together with a roll of adhesive fabric 620 as shown in FIG. 6A. The rollers 630 press the two materials together. The non-adhesive fabric roll 610 has a width A 650 as shown in FIG. 6B. This roll is applied to one edge of the adhesive fabric strip from the roll 620 to form an adhesive/non-adhesive fabric combination 640. The width of the adhesive fabric roll 620 is has a width B 660 as shown in FIG. 6B.

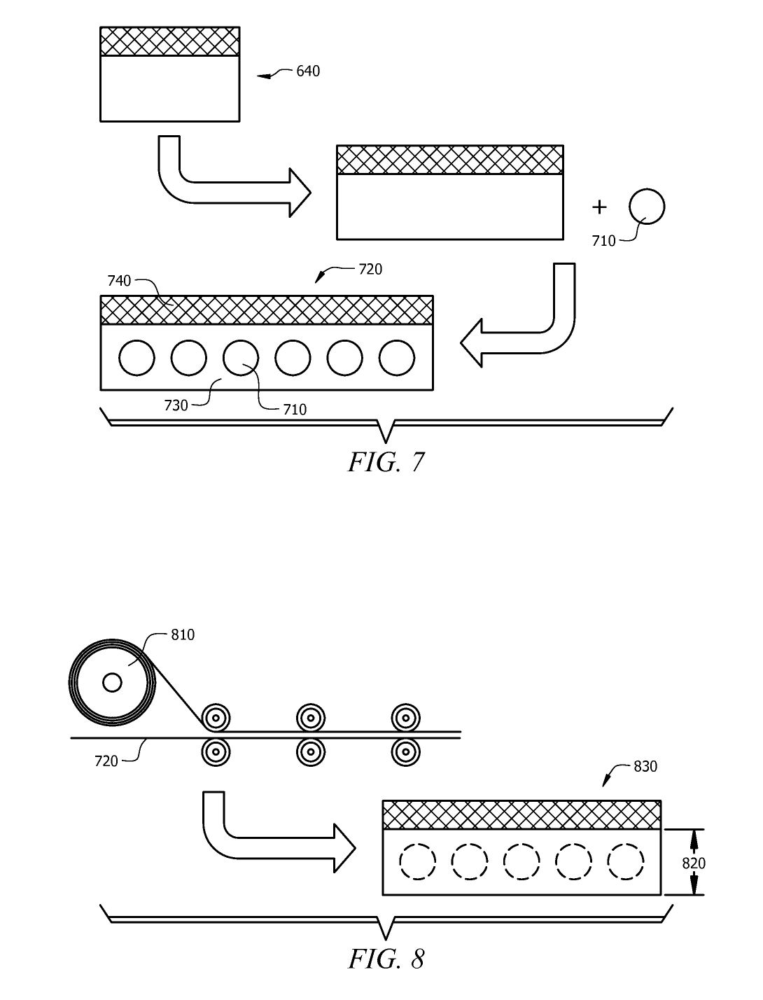

Referring now to FIG. 7, the application of protective cushion pads to the adhesive/non-adhesive fabric combination 640 is illustrated. A high speed pick and place machine is used to apply protective cushion pads 710 to a central portion of the exposed adhesive side of the adhesive fabric 730 located adjacent to where the non-adhesive fabric 740 was applied. The resulting strip 720 has an adhesive portion 730 and a non-adhesive portion 740 and cushion pads 710.

Referring now to FIG. 8, the application of a paper backing to the adhesive portion 730 is illustrated. A white paper roll 810 has a width 820 that is applied over the adhesive portion 730 of the strip 720. The white paper roll contains a nonstick side which is glued to the fabric and a heat activated adhesive side that faces upward. Now the adhesive side of the fabric with adhesive is covered with fabric without adhesive and white paper.

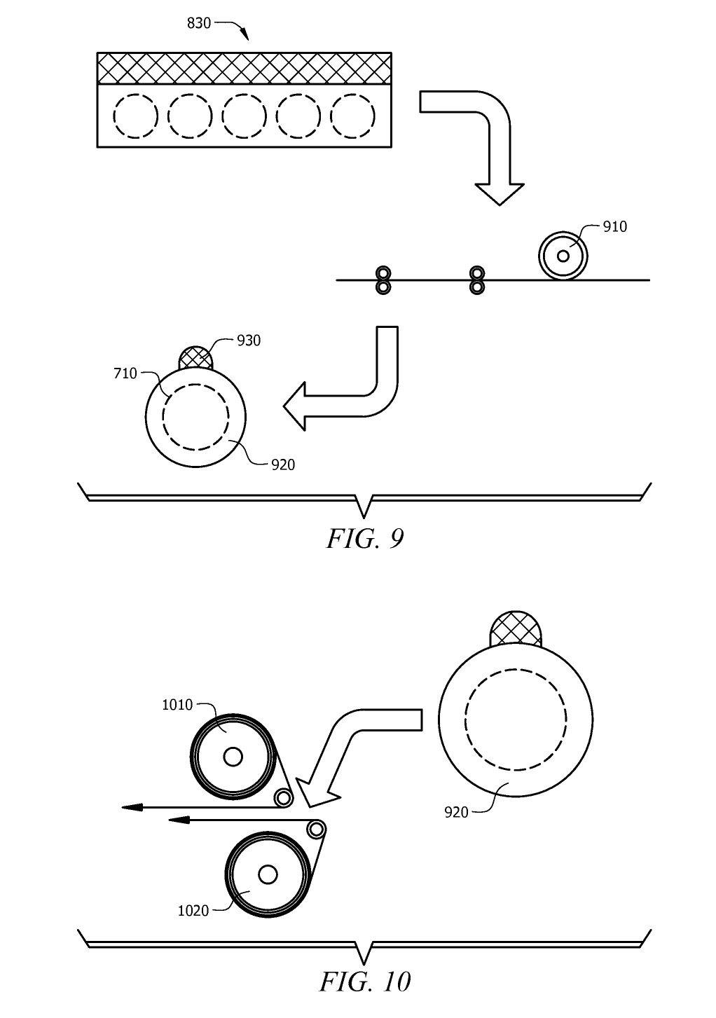

Referring now to FIG. 9, the bandage strip 830 is then placed through a rotary die cutter 910 to produce individual bandages 920. Note that the die cutter is configured to cut a circle around the cushion 710 with a tab 930 remaining where the non-adhesive fabric was applied to the adhesive fabric. Using this process, the bandage 920 now has an adhesive on the circular portion outside of the non-adhesive cushion 710 and has a tab 930 with no adhesive on either side.

Referring now to FIG. 10, the placement of the individual bandages into outer packaging material is illustrated. A high speed pick and place robot arm can be used for this to place the individual bandages 920 on the lower wrapper material 1020 and a upper wrapper material 1010 is placed on top of the bandages. Heat and pressure is then applied to the packaging to form a pouch in which the individual bandages will be held. In the heated roller press, the heat activates the adhesive between the white paper and the wrapping paper and will also activate the adhesive between the two wrapping papers. The adhesive used between the white paper and one side of the wrapping paper can be stronger than the adhesive used between the two wrapping papers and between the tab bandage and the white paper. Thus, the white paper essentially becomes part of the wrapping paper such that when the user peels the pouch to remove the tabbed bandage, the white paper will stay with the wrapping paper because the adhesion strength between the white paper and one side of the wrapping paper is stronger than the adhesion strength between the tabbed bandage and the nonstick side of the white paper. The continuous roll of tabbed bandages can then be perforated. There can be a single tabbed bandage per pouch or multiple bandages per pouch, depending on the needs of the end user. The bandages are then packaged, boxed and sterilized before they are shipped to the end user.

While various embodiments have been described above, it should be understood that they have been presented by way of example only, and not limitation. Thus, the breadth and scope of a preferred embodiment should not be limited by any of the above described exemplary embodiments, but should be defined only in accordance with the following claims and their equivalents. For example, various sizes and shapes of bandages could be utilized, and various packaging materials, glue, paper, etc. could be used without departing from the scope and spirit of the invention.

1. A bandage package for a plurality of adhesive bandages, the bandage package comprising:
a plurality of adhesive bandages, each having an adhesive portion;
a carrier material to which the adhesive portion of each of the plurality of adhesive bandages is removably adhered and wherein the carrier material is packaged loosely between a bottom layer wrapper and a top layer wrapper such that when the top layer wrapper and the bottom layer wrapper are separated by a user, the carrier material with the plurality of adhesive bandages can fall freely from the bottom layer wrapper and the top layer wrapper.

2. The bandage package of claim 1, wherein each of the plurality of adhesive bandages comprises:
a bandage material comprising an adhesive on the adhesive portion of a bottom side for adhering the bandage material to a user and wherein the bandage material comprises a non-adhesive portion on the bottom side that does not adhere to the user or the carrier material; and
a pad attached to the adhesive portion of the bottom side for placement over a wound.

3. The bandage package of claim 2 wherein the non-adhesive portion is configured such that a user can easily grab the non-adhesive portion of the bandage material while wearing a glove.

4. The bandage package of claim 3 wherein the non-adhesive portion is a tab extending outwardly from the adhesive portion.

5. The bandage package of claim 3 wherein the non-adhesive portion extends around an entire perimeter of the adhesive portion.

6. The bandage package of claim 3 wherein the non-adhesive portion extends from a single side of the adhesive portion.

7. The bandage package of claim 3, wherein the bottom layer wrapper and top layer wrapper adhered to each other such that the plurality of adhesive bandages is enclosed.

8. The bandage package of claim 7, wherein the top layer wrapper and the bottom layer wrapper are separable such that when the top layer wrapper is peeled back, the plurality of adhesive bandages is exposed for removal by pulling the non-adhesive portion of each of the plurality of adhesive bandages.

9. The bandage package of claim 4, wherein the top layer wrapper is adhered around a perimeter of the top layer wrapper to the bottom layer wrapper.

10. A method of using a bandage package, the method comprising the steps:
separating a bottom layer wrapper from a top layer wrapper of the adhesive bandage package to allow a carrier material having a plurality of adhesive bandages attached to fall freely to a sterile surface without the user touching the carrier material or the plurality of adhesive bandages, wherein the carrier material is packaged loosely between the bottom layer wrapper and the top layer wrapper;
removing at least one of the plurality of adhesive bandages using sterile fingers;
applying the at least one of the plurality of adhesive bandages to a patient.

11. The method of claim 10 wherein each of the plurality of adhesive bandages comprises:
an adhesive on an adhesive portion of a bottom side;
a non-adhesive portion on the bottom side for facilitating removal from the carrier material;
a pad attached to the adhesive portion of the bottom side for placement over a wound, wherein the carrier material is removably adhered to the adhesive portion adjacent the pad, and wherein the non-adhesive portion of the bottom side does not adhere to the carrier material.

12. The method of claim 11 wherein the top layer wrapper is adhered around a perimeter of the top layer wrapper to the bottom layer wrapper.

13. The method of claim 11 wherein the bottom layer wrapper and the top layer wrapper are adhered to each other such that the plurality of adhesive bandages are completely enclosed prior to step of separating.

14. The method of claim 11 wherein the non-adhesive portion is configured such that a user can easily grab the non-adhesive portion of the bandage material while wearing a glove and wherein the user is wearing the glove while performing the steps of removing and applying.

15. The method of claim 14 wherein the user is not wearing a glove during the step of separating.

16. The method of claim 14 wherein the non-adhesive portion is a tab extending outwardly from the adhesive portion of the bandage material.

17. The method of claim 16 wherein the non-adhesive portion extends around an entire perimeter of the adhesive portion of the bandage material.

18. An adhesive bandage, the adhesive bandage comprising:
a bandage material having an adhesive on an adhesive portion of a bottom side for adhering the bandage material to a user and wherein the bandage material has a non-adhesive portion on the bottom side that does not adhere to the user;
a pad attached to the adhesive portion of the bottom side for placement over a wound;
a carrier material to which the adhesive portion is removably adhered wherein the non-adhesive portion of the bottom side does not adhere to the carrier material.

19. The adhesive bandage of claim 18 wherein the carrier material is packaged loosely between a bottom layer wrapper and a top layer wrapper such that when the top layer wrapper and the bottom layer wrapper are separated by a user, the carrier material with attached bandage material can fall freely from the bottom layer wrapper and the top layer wrapper.

20. The adhesive bandage of claim 19 wherein the non-adhesive portion is a tab extending outwardly from the adhesive portion of the bandage material.
